(19) United States
(12) Patent Application Publication
(10) Pub. No.: US 2016/0183976 A1
(43) Pub. Date: Jun. 30, 2016

(54) Cannula-needle with protective member

(71) Applicant: DELTA MED S.P.A., VIADANA (IT)

(72) Inventors: Alessandro Bertoli, San Giovanni in Croce (CR) (IT); Alessandro Balboni, San Giorgio di Mantova (MN) (IT)

(73) Assignee: DELTA MED S.P.A., Viadana (IT)

(21) Appl. No.: 14/653,792

(22) PCT Filed: Dec. 16, 2013
(86) PCT No.: PCT/IB2013/060978

§ 371 Date: Jun. 18, 2015

(30) Foreign Application Priority Data
Dec. 20, 2012 (IT) MO2012A000309

(51) Int. Cl. A61B 17/34

(52) U.S. Cl. CPC A61B 17/3496

(57) ABSTRACT

A cannula-needle with a protective member includes a needle holder that holds a needle, which is designed to be coaxially coupled with a cannula for making an injection into a blood vessel, and which has a stem having a longitudinal axis, a distal tip and a transverse engagement member in the proximity of the tip; a cannula holder, with the cannula extending from a distal end thereof, and with an inner cavity mating with the needle holder and having inner walls formed at the opposite proximal end; a protective member protecting the needle tip and movable from a disabled position to an enabled position, in which it is irreversibly placed beyond the needle tip after use of the cannula-needle, the protective member being housed between the inner walls of the inner cavity in the disabled position.

### FIELD OF THE INVENTION

The invention relates to a cannula-needle with a protective member, which is generally used to allow health care operators to handle a cannula-needle with the utmost safety without any risk of being accidentally pricked.

### BACKGROUND ART

A number of medical apparatus are known which comprise a needle for making injections into blood vessels of patients, and which are equipped with protective devices to prevent operators from being accidentally injured after use.

Particularly, these safety devices are used to prevent transmission of infectious diseases through accidental punctures from a patient to health care operators who treat him/her and provide the required therapy.

Typically, prior art protective devices consist in specially shaped springs or ratchet gears, which are designed to be disabled when the needle is used, and are enabled after needle use, i.e. once the needle has been extracted from the blood vessel of the patient after the injection.

More in detail, prior art protective devices are arranged to slide on a needle stem and irreversibly come to a position in which they entirely cover the needle tip.

These devices are placed in special housing elements, typically in the form of box-like shells, which contain them and allow the operators to grab and slide them along the needle stem from the disabled position to the active protection position.

Further prior art protective devices are designed for automatic actuation during needle withdrawal from the blood vessel of the patient.

Also in this case, the safety device is held within a box-like shell which is arranged, like in the previous cases, between a proximal end of the needle and the needle-holder, and hence is an additional element that is always present on the apparatus, both during and after use thereof.

The above described prior art suffers from certain drawbacks.

A first drawback consists in that these protective devices always require a box-like shell that contains them.

This will considerably increase the manufacturing costs for apparatus having protective devices, both because they require a special shell to contain the safety device and because safety devices must be assembled and inserted into their respective shells.

A second drawback is that, even when shells have small sizes, they are still provided on the needle stem as additional bodies, thereby limiting handling freedom by health care operators.

It should be noted that the needles that are typically used in the medical field to make injections into the blood vessels of patients are very small and that the operators are required to wear protective gloves that limit finger sensitivity and hence, grip safety.

Therefore, the presence of an additional element is an additional restriction to freedom of movement, as mentioned above.

### DISCLOSURE OF THE INVENTION

The invention has the object to improve the prior art.

In one aspect, the invention relates to a cannula-needle with a protective member according to the features of claim 1.

The invention achieves the following advantages:
providing a cannula-needle that contains the protective member therein and avoids the need for additional container bodies;
improving handling of the cannula-needle, as no container for the protective member is interposed between the needle-holder and the needle;
affording automatic actuation of the protective member during withdrawal of the needle from the cannula, once the latter has been inserted into a blood vessel of a patient.

### BRIEF DESCRIPTION OF THE DRAWINGS

Further features and advantages of the invention will be more readily apparent upon reading of the detailed description of preferred non-exclusive embodiments of a cannula-needle with a protective member, which is shown as a non-limiting example in the annexed drawings, in which:

FIG. 1 is a highly schematic and magnified broken view of a longitudinal section of a cannula-needle of the invention, in a configuration of use, as taken along a plane I-I of FIG. 14;

FIG. 2 is a view of the cannula-needle of FIG. 1, in a later configuration of use;

FIG. 3 is a detail view of a section of a needle stem upon which a tip protection member is mounted, in a disabled and cannula holder-free configuration, for clarity;

FIG. 4 is a view of the needle stem section of FIG. 3, in an enabled configuration of the protective member;

FIG. 5 is a side view of the protective member of the invention;

FIG. 6 is a perspective view of the protective member from a first point of view;

FIG. 7 is a perspective view of the protective member from a second point of view, opposite to that of FIG. 6;

FIGS. 8a to 8d are schematic, smaller-scale views of the steps from a disabled state to an enabled state of the protective member of FIG. 1;

FIG. 9 is a perspective view of a needle holder in which the protective member is in the enabled configuration;

FIG. 10 is a longitudinal sectional view of a cannula with a protective member mounted therein according to the invention, as taken along a plane X-X of FIG. 14;

FIG. 11 is a longitudinal sectional magnified view of a detail of the cannula of FIG. 10, as taken along a plane XI-XI of FIG. 13.

FIG. 12 is a further magnified view of the protective member in an enabled configuration;

FIG. 13 is a cross-sectional view of the cannula of FIG. 10, as taken along a plane XIII-XIII of FIG. 10;

FIG. 14 is a general, slightly smaller view of the cannula-needle of FIG. 1, in an assembled, ready-for-use configuration.

### DETAILED DESCRIPTION OF A PREFERRED EMBODIMENT

Referring to the figures, numeral 1 generally designates a cannula-needle, which is typically designed for use to make injections into a blood vessel of a patient.

The cannula needle 1 comprises a needle holder 2 which supports a needle 3 having a longitudinal axis “A” and is designed to be coaxially inserted into a flexible cannula 4, to temporarily impart flexural strength thereto, and act as an introducer for the blood vessel to be introduced into the blood vessel without bending.

The cannula 4 is supported at the distal end by a cannula holder 5 and extends therefrom outwards.

The needle 3 forms a tip 3a at the distal end of the stem 3b and, proximate thereto, a transverse engagement member, namely a raised boss 9.

The cannula holder 5 has an axial inner cavity 6 formed therein, which is delimited by walls 7 and in which a protective member 8 is designed to be precisely received and removably retained.

This protective member 8 consists of a sheet-like body, which is typically made with a metal material, such as harmonic steel, and is shaped to form a proximal wall 10 transverse to the stem 3b of the needle 3 and two arms 11 and 12 extending from two opposite and folded edges 10a of the wall 10, and face toward the tip 3a.

The arm 11 is formed in such a manner that it may maintain a spontaneously oblique position relative to the arm 12, to tend and converge toward the latter and, as a result, toward the stem 3b of the needle 3.

Furthermore, the arm 11 forms a second wall 13, at the end opposite to the one that is connected to the wall 10, which is folded toward the arm 12 and is divided into two successive sections 13a and 13b, slightly bent relative to each other.

A first opening 14 is formed in the wall 10, which has a circular perimeter and is designed for the stem 3b of the needle 3 to slide therethrough.

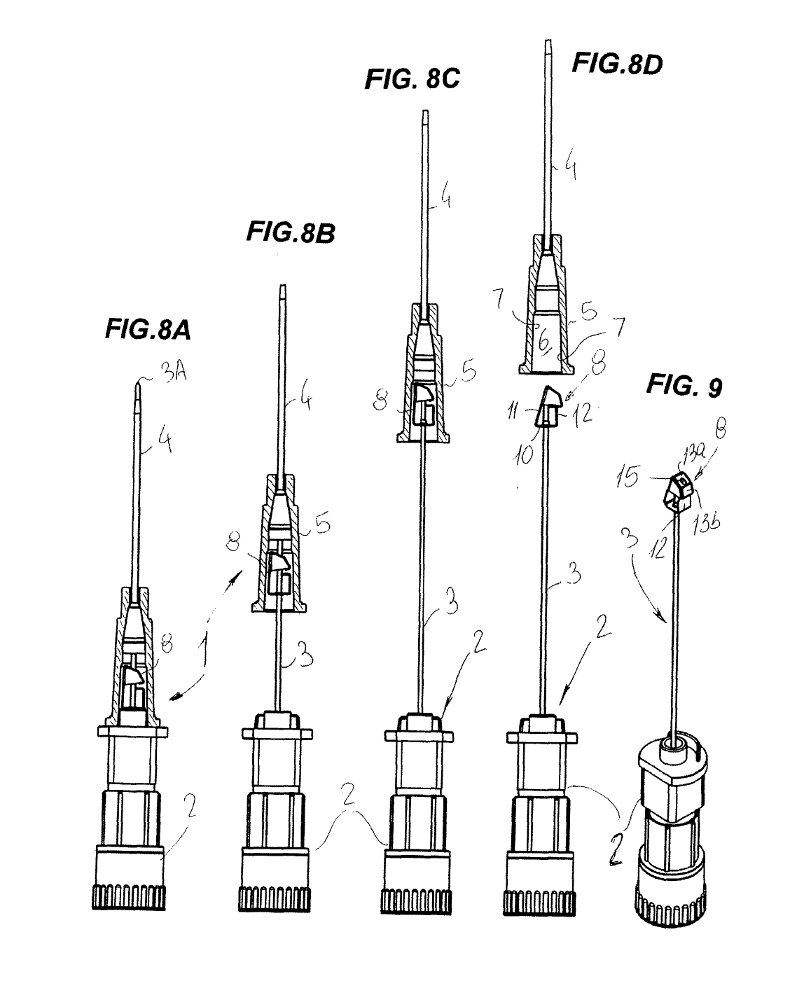

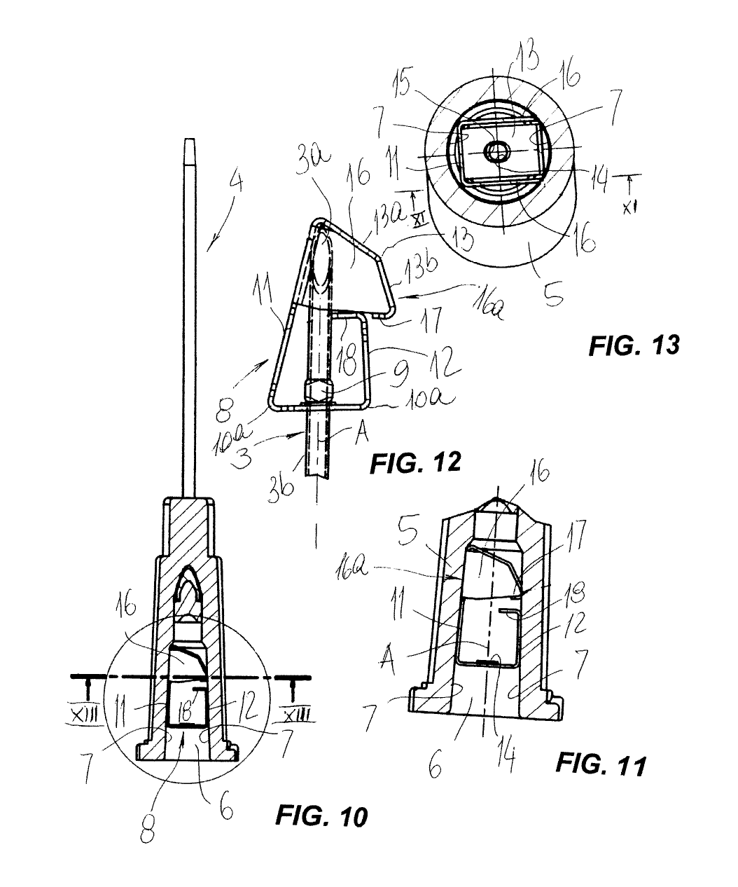

A second opening 15 is formed in the section 13a of the second wall, which is also designed for the stem 3b to extend therethrough, when the protective member 8 is in a disabled configuration, as shown in FIGS. 3, 8a, 8b, 8c.

The arm 11 also forms two lateral partitions 16 which are folded perpendicular thereto and form with the second wall 13 adjacent thereto, a sort of box-like head 16a, which nevertheless has an entirely open side facing the wall 10.

The second section 13b is formed with a lip 18 at its free end, which is folded toward the arm 11 and whose purpose is described below.

The arm 12 is substantially straight and stationary with respect to the proximal wall 10 and is substantially perpendicular thereto.

The arm 12 has a free distal end that forms a second lip 18 folded toward the arm 11 and hence toward the stem 3b of the needle 3.

It shall be noted that the diameter of the opening 14 is slightly smaller than the diameter of the boss 9 and for this reason, the boss 9 cannot pass through it when the needle 3 slides relative to the protective member 8.

Conversely, the second opening 15 has a slightly larger size than the outside diameter of the boss 9, such that the latter can pass through it when the needle 3 slides relative to the protective member 8.

The protective member 8 is normally retained in the disabled configuration within the cavity 6, typically by adhesion between the edges of the lateral partitions 16 and the walls 7, as shown in greater detail in FIG. 13.

When the cannula-needle 1 is not used, the stem 3b extends through both openings 14 and 15 and transversely pushes the arm 11, thereby forcing it to bend toward the wall 7 to a biased position substantially parallel to the arm 12.

This state is shown in schematic detail in FIGS. 1, 3 and more generally in FIGS. 8a, 8b, 8c.

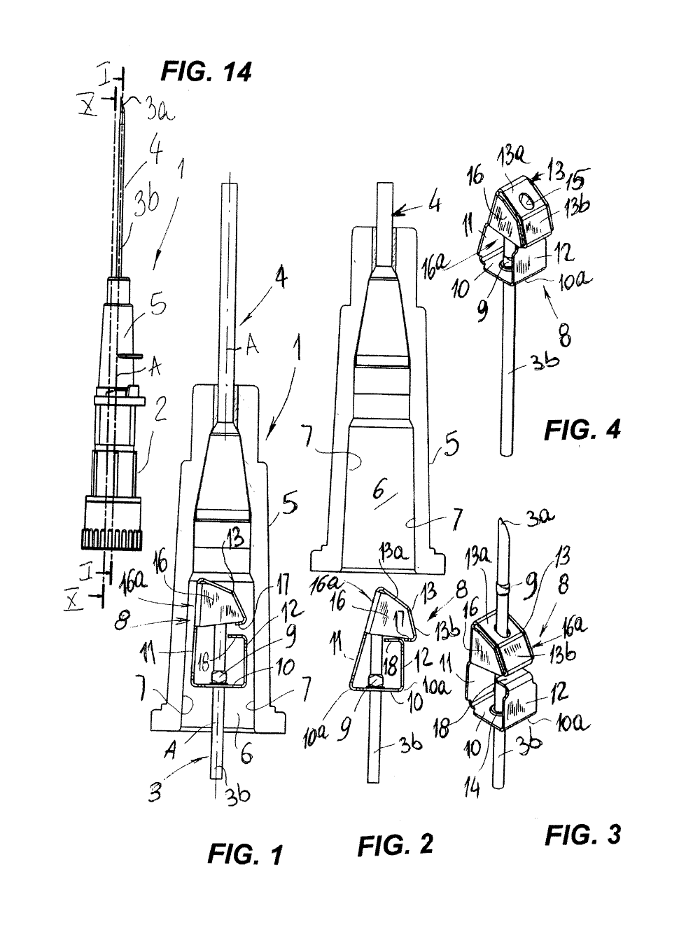

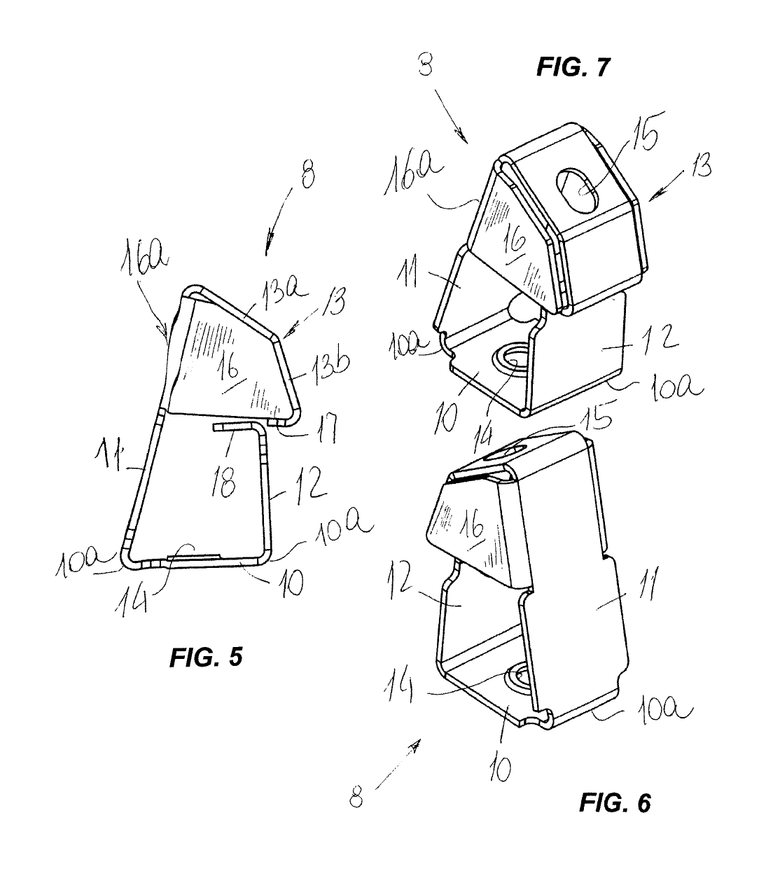

After use of the cannula-needle 1, when the protective member 8 is in the enabled position, the arm 11 takes its natural oblique position relative to the proximal position 10 and the second wall 13 comes before the tip 10a, as shown in FIGS. 2 and 12.

The operation is as follows: when the cannula-needle 1 is in a non-use or preparation-for-use configuration, it is in the state as shown in FIG. 8a.

More in detail, the protective member 8 is inserted in the axial cavity 6 and is retained therein by adhesion between the partitions 16 and the inner walls 7.

The needle 3 extends through both walls 10 and 13 of the protective member 8, after passing through the openings 14 and 15.

It shall be noted that the second opening 15 is slightly elliptical for alignment with the first opening 14 in this disabled state of the protective member 8, although the second wall 13 thereof is oblique to the proximal wall 10: this aligned state, which allows the needle 3 to slide, is shown in greater detail in FIG. 13.

Once the health care operator has made an injection with the cannula-needle 1, by introducing it into a blood vessel of a patient, he/she withdraws the needle 3 from the cannula 4 by a withdrawal operation, i.e. by maintaining the cannula 4 in the blood vessel of the patient and simultaneously pulling out the needle in the proximal direction 3, while holding the needle holder 2.

In this pull-out operation, the stem 3b of the needle 3 progressively slides within the two openings 14 and 15 (see FIG. 8b) until the boss 9 stops against the peripheral edge of the first opening 14, after passing through the second opening 15: this condition is shown in FIG. 8c.

As the pull-out operation continues, the operator overcomes the adhesion force that retains the protective member 8 in the cavity 6 and progressively pulls it out with the needle 3, until it is entirely withdrawn from the cavity 6: this condition is shown in FIG. 8d.

As the tip 3a of the needle 3 is pulled out of the protective member 8, moves beyond the second opening 15 and disengages therefrom, the arm 11 is instantaneously restored into its oblique position and the second opening 15 is finally and irreversibly out of alignment with the first opening 14 and with the tip 3a, which is entirely and automatically covered by the first section 13a of the second wall 13 (see FIG. 12), whereby the operator is safeguarded against accidental pricks as he/she completes withdrawal and later disposes of the needle 3.

As shown in FIG. 12, in the enabled configuration the protective member 8 is slightly inclined to the axis “A” of the stem 3b of the needle 3 and the second lip 18 abuts the stem 3b, thereby adding stability to the position of the protective member 8 on the needle 3.

The invention has been found to fulfill the intended objects.

The invention so conceived is susceptible to changes and variants within the inventive concept.

Also, all the details may be replaced by other technical equivalent elements.

In its practical implementation, any material, shape and size may be used as needed, without departure from the scope as defined by the following claims.

The invention claimed is:

1. A cannula-needle (1) comprising:
a needle holder (2) that holds a needle (3), said needle being designed to be coaxially coupled with a cannula (4) for making an injection into a blood vessel and having a stem (3b) with a longitudinal axis (A), a distal tip (3a) and a transversal engaging member (9) fitted in a proximity of said distal tip (3a);
a cannula holder (5), from one end of which said cannula (49) extends, the cannula holder defining an inner cavity (6) at an opposing end for coupling with said needle holder (2), said inner cavity having inner walls (7); and
a protective member (8) of said needle tip, which can be moved from a deactivated position to an irreversible activated position after use of the cannula-needle (1) wherein said protective member is placed over the distal tip (3a),
wherein said protective member (8) in said deactivated position is retained in a removable way among said inner walls (7) of said inner cavity (6).

2. The cannula-needle as claimed in claim 1, wherein, between said inner cavity (6) and said protective member (8), a removable retention member (16) of said protective member (8) is interposed, said removable retention member (16) being configured to restraint said protective member (8) in said deactivated position and to release said protective member (8) toward said activated position.

3. The cannula-needle as claimed in claim 2, wherein said protective member (8) comprises a flat body which shapes:
a transversal proximal wall (10) to said needle, wherein a first passage opening (14) for said stem (3b) is defined having a blocking perimeter of said transversal engaging member (9);
a first and a second arm (11, 12) which extend from said proximal wall (10) toward said distal tip (3a); and
at least said first arm (11) shaping a second wall (13) transversally fitted with respect of said needle (3), wherein a second passage opening (15) of said stem (3b) is defined, said second passage opening (15) in said deactivated position being aligned with said first passage opening (14) and in said activated position being misaligned from said first passage opening (14).

4. The cannula-needle as claimed in claim 3, wherein said retention member comprises a couple of lateral surfaces (16) that extend from at least one of said first and second arms (11, 12) and converge toward said second wall (13), said lateral surfaces (16) having respective gripping edges on said inner walls (7) of said inner cavity (6).

5. The cannula-needle as claimed in claim 3, wherein said first arm (11) in said activated position is oblique with respect of said transversal proximal wall (10).

6. The cannula-needle as claimed in claim 3, wherein said second arm (12) is perpendicular to said transversal proximal wall (10).

7. The cannula-needle as claimed in claim 3, wherein said protective member (8) is configured to be slidingly withdrawn from said inner cavity (6) during retraction of said needle (3), differently from said removable retention member (16) when said transversal engaging member (9) is blocked against said blocking perimeter of said first passage opening (14).
